(19) United States
(12) Patent Application Publication
(10) Pub. No.: US 2024/0212247 A1
(43) Pub. Date: Jun. 27, 2024

(54) SOFTWARE APPLICATION FACILITATING A METHOD OF SELF-DEVELOPMENT

(71) Applicant: Tanya Jean Shewfelt, Toronto (CA)

(72) Inventor: Tanya Jean Shewfelt, Toronto (CA)

(21) Appl. No.: 18/069,493

(22) Filed: Dec. 21, 2022

(51) Int. Cl. G06T 13/40; A61B 5/16

(52) U.S. Cl. CPC G06T 13/40; A61B 5/16

(57) ABSTRACT

A self-development method that is configured to be facilitated by a software application wherein the present invention the user is taught about a plurality of self-knowledge tools and guided therethough. The present invention further includes providing of a graphical interface environment during utilization thereof. The graphical interface environment includes a plurality of three-dimensional avatars that include but are not limited to a personal three-dimensional avatar and a wisdom guide avatar. The software application of the present invention facilitates data collection throughout utilization thereof wherein the data collection is compiled for analysis in a personal database. Data input is collected by either voice input or text input. The personal database is populated utilizing the tools within the software and subsequent activities and/or actions are required to be performed by the user in order to drive improvement in the category.

### FIELD OF INVENTION

The present invention relates generally to individual psychological development, more specifically but not by way of limitation, a software application that is operable to facilitate self-development of a user wherein the present invention creates a knowledge database on the user and provides guidance based on the data stored therein.

### BACKGROUND

Self-psychology, an offshoot of Freuds's psychoanalytic theory, forms much of the foundation of contemporary psychoanalysis as the first large psychoanalytic movement recognizing empathy as an essential aspect of the therapeutic process of addressing human development and growth. Self-psychology theory focuses on the development of empathy towards the patient and the exploration of fundamental components of healthy development and growth. Professionals may use self-psychology theory in part to help people consider how their early experiences may contribute to the formation of their sense of self. Additionally, exploration and development of categories such as but not limited to self-esteem and self-trust are an important part of self-psychology

In self psychology, the individual is understood to be the center of one's psychological universe. If a child's developmental environment is appropriate, a healthy sense of self will typically develop and generally the individual will be able to maintain consistent behavior patterns allowing an individual to self-regulate and self-soothe throughout life. When individuals are not able to develop a healthy sense of self, they may tend to rely on others in order to get needs met. These others are called self-objects. Self-objects are a normal part of the developmental process. Children need self-objects because they are incapable of meeting all of their own needs, but over the course of healthy development, self-objects become internalized as individuals develop the ability to meet their own needs without relying on external others. Self-psychology can help provide a clearer and more comprehensive understanding of one's own self and is beneficial for many as it provides an education and tools in order to achieve the objective. Typically, this type of therapy is performed by a professional therapist which many times is not covered by health insurance and can be cost prohibitive for many that could benefit from the therapy.

It is intended within the scope of the present invention to provide a software application operable to facilitate self-development of a user without the requirement for a professional therapist.

### SUMMARY OF THE INVENTION

It is the object of the present invention to provide a software application that is operable to facilitate self-development of a user wherein the present invention provides a plurality of tools for users to employ to perform self-development.

Another object of the present invention is to provide a self-development method configured to facilitate development of a user of the present invention wherein the present invention includes creation of a personal database that is dynamic in content.

A further object of the present invention is to provide a software application that is operable to facilitate self-development of a user wherein the present invention includes generation and use of a three-dimensional avatar.

Still another object of the present invention is to provide a self-development method configured to facilitate development of a user of the present invention wherein the development tools provided by the present invention include but are not limited to self-esteem, self-respect and self-healing.

An additional object of the present invention is to provide a software application that is operable to facilitate self-development of a user wherein the present invention further includes utilization of simulated environments during navigation of the software.

Yet a further object of the present invention is to provide a self-development method configured to facilitate development of a user of the present invention wherein the software employs a wisdom guide to accompany the individual's avatar during utilization of the tools of the software application of the present invention.

Another object of the present invention is to provide a software application that is operable to facilitate self-development of a user wherein the present invention includes a detachment tool.

An alternate object of the present invention is to provide a self-development method configured to facilitate development of a user of the present invention wherein the software application provides a generated virtual environment that is a protective space.

Still a further object of the present invention is to provide a software application that is operable to facilitate self-development of a user wherein the self-knowledge tool of the present invention includes a plurality of categories of tools.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is an outline of the method of the present invention; and

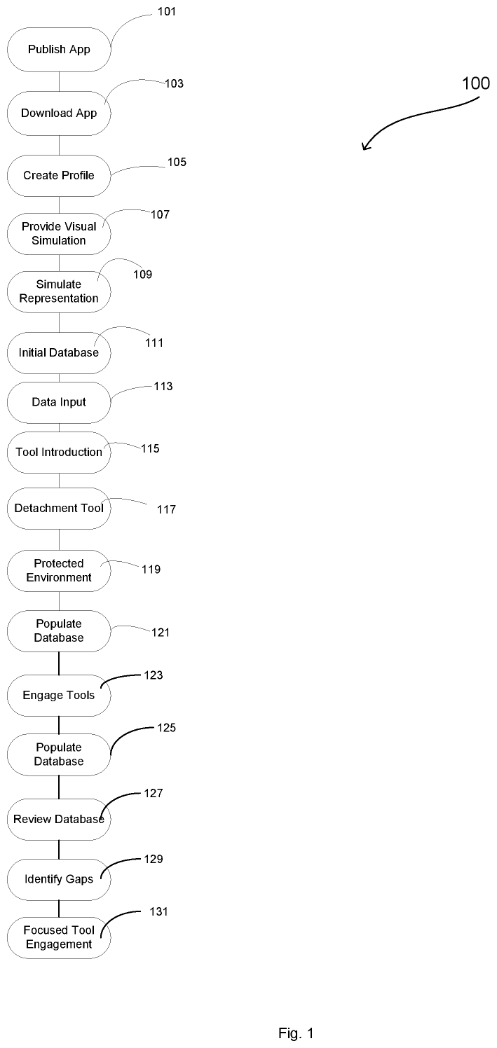

FIG. 2 is an outline of utilization of the self-knowledge tools of the present invention.

### DETAILED DESCRIPTION

Referring now to the drawings submitted herewith, wherein various elements depicted therein are not necessarily drawn to scale and wherein through the views and figures like elements are referenced with identical reference numerals, there is illustrated a self-development method 100 constructed according to the principles of the present invention.

An embodiment of the present invention is discussed herein with reference to the figures submitted herewith. Those skilled in the art will understand that the detailed description herein with respect to these figures is for explanatory purposes and that it is contemplated within the scope of the present invention that alternative embodiments are plausible. By way of example but not by way of limitation, those having skill in the art in light of the present teachings of the present invention will recognize a plurality of alternate and suitable approaches dependent upon the needs of the particular application to implement the functionality of any given detail described herein, beyond that of the particular implementation choices in the embodiment described herein. Various modifications and embodiments are within the scope of the present invention.

It is to be further understood that the present invention is not limited to the particular methodology, materials, uses and applications described herein, as these may vary. Furthermore, it is also to be understood that the terminology used herein is used for the purpose of describing particular embodiments only, and is not intended to limit the scope of the present invention. It must be noted that as used herein and in the claims, the singular forms “a”, “an” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

References to “one embodiment”, “an embodiment”, “exemplary embodiments”, and the like may indicate that the embodiment(s) of the invention so described may include a particular feature, structure or characteristic, but not every embodiment necessarily includes the particular feature, structure or characteristic.

Now referring to the Figures submitted as a part hereof, the self-development method 100 is facilitated by a software application wherein the software application is configured to be operated on computing devices such as but not limited to smartphones and computers. In step 101, the operator of the self-development method 100 published the software application of the present invention to an application repository, website or computing device so as to make available to potential users via conventional communication protocols. Step 103, the users will download the software application of the present invention. In step 105, ensuing download of the software application of the present invention, the users will create a profile. During creation of a profile, the software application facilitates generation of a three-dimensional avatar wherein the avatar has been created from an uploaded photograph of the user. Step 107, the user is provided with a graphical simulated environment wherein the environment includes a graphical image of a door wherein the door includes instructions thereon to knock. The user will virtually knock and is subsequently requested to create an audio password wherein the user's voice is employed for password protection, a wisdom guide as well as avatars created during utilization of the software of the present invention.

In step 109, the graphical environment presented to the user of the software application of the present invention includes presentation of a simulation of an inner human body. During presentation of the inner human body, a simulation of the avatars' body becoming opened by way of a zipper is presented to the user and the wisdom guide is presented to the user wherein the wisdom guide employs the voice of the user previously captured in step 107. In step 111, the commencement of the personal database, referred to as “book of me” in the software application begins. It should be understood within the scope of the present invention that the personal database dynamically captures data about the user throughout utilization of the method of the present invention. Data is consistently input and scenarios presented to the user based on recent information received in the personal database.

In step 113, as previously stated, data input is consistently executed during use of the software wherein the data input can be executed through voice or keyboard unput. Data input is collected and analyzed through all stages of execution of the present invention. In step 115, the software application provides a menu of tools that can be utilized by the users. In order to introduce tools in a productive order, the software application will generate questions for which the user must supply answers. Situational analysis questions are provided to the user wherein these questions are followed by personal reflection questions. Based on these answers, the software application will create avatars of historical figures relevant to the answers provided by the user. A virtual store is available for users to buy items throughout utilization of the software application. An emotional bank resides within the software application wherein the emotional bank is a virtual bank that allows a deposit of a virtual token upon completion of a self-relationship task. Tracking of deposits within the self-relationship task categories, discussed further herein, provide a tracking method for identification of self-relationship tasks that require more development by the user.

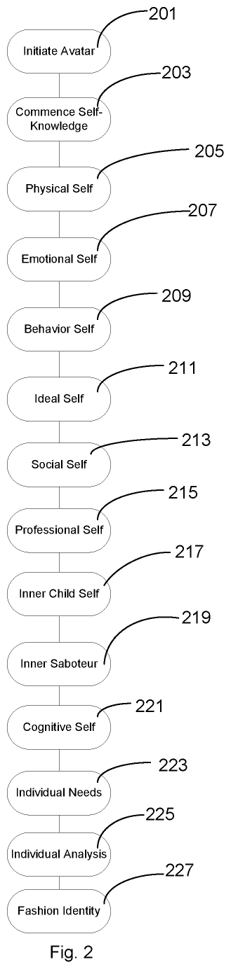

Step 115 can further includes instructional avatars for subjects such as art, nature, photography and film. In step 117, the user is introduced to a detachment tool. The detachment tool is utilized to perform a self-analysis for individuals that are potentially too self-focused and need to take a proverbial step back and look at themselves. In one contemplated embodiment of the present invention this can be executed from a football coach avatar as well as an out of body type view. In step 119, if deemed required, the software application will generate a virtual representation of a protected environment for a user. The protected environment is selected by a user and represents the inner world of the user wherein the wisdom guide avatar provides instruction on creation of an inner protected environment. In step 121, the personal database is populated with data collected in the aforementioned steps. It should be understood that data collection is not a discrete step but ongoing as data is input into the software application. Step 123, a user will engage in at least one of the self-knowledge tools provided by the software application. The self-knowledge tools facilitate a process further discussed herein and outlined in FIG. 2. In step 125, the personal database is populated as the various self-knowledge tools are employed by the user. Step 127, the software application of the present invention provides a review of the personal database for the user. In step 129, the software application presents categories for a user to developed based on the aforementioned tracking system of virtual coins being deposited into the emotional back and the associated category. Furthermore, the presented categories can be determined by the software application through observed deficiencies in responses within the self-knowledge tools. Step 131, the user will focus on utilizing the identified self-knowledge tools as identified through engagement in the self-development method 100 wherein the focused self-knowledge tool utilization results from previous responses and recordation thereof in the personal database and is operable to provide improvement through analysis of data within the personal database and a directional shift with a positive change in at least one of the self-knowledge categories. Integrated to step 131 will be an activity and/or study that the user must complete and subsequently enter data thereabout into the personal database. It should be understood within the scope of the present invention that the software application will assign a development value for each of the categories discussed herein. The development value identifies in which categories the user excels and in which categories the user will be guided for improvement therein. As the user focuses on a category, the activities and/or exercises provided by the software application will result in an increase in the development value of that category.

Now referring to FIG. 2 submitted as a pert hereof, an outline of the self-knowledge process as facilitated by the self-development method 100 is diagrammed therein. In step 201, the software application of the self-development method 100 will facilitate creation of an avatar representative of the user. It should be understood, that a single or multiple avatar could be generated by the software application at any step within utilization of the software application of the self-development method 100. Step 203, the user will initiate the self-knowledge process of the present invention. It should be understood within the scope of the present invention that the user can complete all of the self-knowledge categories, a few or just one and order can be executed as desired based on user's preference and/or needs as identified by the software application through data collected in the personal database. In a preferred embodiment of the present invention, the wisdom guide avatar graphically accompanies the user during the self-knowledge process. In step 205, the user is present with a physical self category of the self-knowledge process. In this step, the software application generates a user avatar and provides animation of a submarine within the internals of the user avatar. The submarine is controllable by the user wherein the submarine graphic can be directed to any body part and upon stopping at the body part information thereon is provided to the user. Additionally, the user can input any data about the body part such as but not limited to exercise data and health data wherein this information is stored in the personal database.

Step 207, the user is presented with an emotional self category as part of the self-knowledge process. In this step the user is guided through an emotional regulation simulation that teaches the user about emotional management, identification of emotions, breathing and other parameters such as but not limited to judgement. In step 209, the user is presented with a behavior self category. In this step, the user is presented with questions regarding behavior in various scenarios as wells as questions regarding personality traits. All answers to the questions are recorded into the personal database. Step 211, an ideal self category is presented to the user. In the ideal self category a user is guided through an ideal self simulation wherein the user can input parameters such as but not limited to aspirations, dreams and goals.

In step 213, the software application presents a social self category to the user. In this simulation presented to the user, the user is prompted to send questionnaires to family and friend about their social habits wherein the data collected from the family and friends of the user is stored in the personal database. Step 215, a professional self category is presented to the user. This category prompts questions to the user about the user's professional work habits, goals and objectives wherein the answers thereof are stored in the personal database. In step 217, the user is presented with inner child self category. In this step the user is present with questions about their childhood and events thereof wherein the data is divided within certain age brackets and subsequently stored in the personal database. Step 219, the software application of the self-development method 100 presents an inner saboteur category to the user. In this step, the user is prompted to enter data regarding destructive thoughts wherein these thoughts subsequently create generation of avatars depicting the thoughts. Furthermore, the wisdom guide avatar is presented to the user wherein the wisdom guide avatar suggests alternative thoughts and processes to counter the saboteur thoughts.

In step 221, the self-development method 100 presents a cognitive self category to the user. In this step the user is presented with questions focused on determining the cognitive ability of the user. Through analysis of the inputted data the software application identifies potential cognitive gaps and records this data in the personal database. Step 223, the user is presented with a individual needs category. In this step the user's needs are divided into physical, emotional, relationship and psychological. Questions and/or prompts are provided to the user on each need category wherein responses thereto are recorded in the personal database and the software application renders an assessment of the user's needs. In step 225, the user is prompted for an individual analysis. The individual analysis utilizes questions and prompts to collect data on what has happened to the user in their life by inquiring about various life events. Analysis for patterns, trauma and other parameters is executed wherein this data is stored in the personal database. Step 227, the user is presented with a fashion identity category. In this step, the user employs the software application to dress their avatar numerous times wherein the user can select from a multitude of clothing items to dress the avatar. Questions and prompts are provided to the user about clothing preferences and the answers thereto are recorded in the personal database.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding description is, therefore, not intended to be limited to the specific forms set forth herein, but on the contrary, it is intended to cover such alternatives, modifications, and equivalents, as can be reasonably included within the spirit and scope of the invention.

What is claimed is:

1. A self-development method facilitated by a software application wherein the self-development method drives personal understanding and behavioral improvement and wherein the self-development method comprises the steps of:
providing the software application, said software application being stored on a computing device, wherein the computing device is accessible via conventional communication protocols;
downloading the software application, wherein users will download the software application onto a remote computing device;
creating a profile, wherein the user will create a profile for use of the self-development method, wherein during creating a profile a graphical personal three-dimensional avatar is produced to represent the user;
providing a visual simulation, wherein the user is provided with a graphical interface of a simulated environment;
providing a wisdom guide avatar, wherein the wisdom guide avatar is a three-dimensional avatar that is graphically presented adjacent to the personal three-dimensional avatar during utilization of the software application;
establishing a personal database, wherein the wisdom guide avatar provides generation of questions to be answered by the user and wherein the answers are recorded into the personal database;
introducing a plurality of self-knowledge tools, wherein the software application provides a plurality of categories of self-knowledge tools to the user;
selecting at least one self-knowledge tool, wherein the user selects a self-knowledge tool from the plurality of categories of self-knowledge tools;
completing the selected self-knowledge tool, wherein the user is presented with questions during engagement with the selected self-knowledge tool;
recording answers to the questions from the selected self-knowledge tool in said personal database;
establishing a development value for the selected self-knowledge tool, wherein the development value is recorded in said personal database; and
identifying a self-knowledge tool for a user to engage to drive improvement therein based on the development value.

2. The self-development method facilitated by the software application operable to drive improvement in personal understanding as recited in claim 1, and further including a step of providing a physical self category tool of self-knowledge, wherein the physical self category of self-knowledge includes an ability to select a body part of a user and enter information thereabout into said personal database.

3. The self-development method facilitated by the software application operable to drive improvement in personal understanding as recited in claim 2, wherein the user profile avatar is created through uploading of a picture of the user.

4. The self-development method facilitated by the software application operable to drive improvement in personal understanding as recited in claim 3, and further including a step of providing an emotional self category tool of self-knowledge, wherein the user is guided through an emotional regulation simulation that teaches the user about emotional management and identification of emotions.

5. The self-development method facilitated by the software application operable to drive improvement in personal understanding as recited in claim 4, and further including a step of providing a behavior self category tool of self-knowledge, wherein the user is provided a list of questions about behavior and personality traits and wherein answers thereto are recorded in said personal database.

6. The self-development method facilitated by the software application operable to drive improvement in personal understanding as recited in claim 5, and further including a step of providing an ideal self category tool of self-knowledge, wherein the user is guided through an ideal self simulation wherein the user can inputs parameters regarding aspirations, dreams and goals.

7. The self-development method facilitated by the software application operable to drive improvement in personal understanding as recited in claim 6, and further including a step of providing an ideal social category tool of self-knowledge, wherein the user and at least one associate thereof is presented with questions regarding social behavior of the user, wherein answers to the questions about social behavior are recorded into said personal database and employed to determine a development value of the ideal social category tool of self-knowledge.

8. The self-development method facilitated by the software application operable to drive improvement in personal understanding as recited in claim 7, and further including a step of providing an inner child category tool of self-knowledge, and further including a step of providing an inner child category tool of self-knowledge, wherein the user is presented with questions regarding events of childhood, wherein answers to the questions about inner child category are recorded into said personal database and employed to determine a development value of the inner child category tool of self-knowledge.

9. The self-development method facilitated by the software application operable to drive improvement in personal understanding as recited in claim 8, and further including a step of providing an inner saboteur category tool of self-knowledge, wherein the user inputs data regarding destructive thoughts into said personal database.

10. The self-development method facilitated by the software application operable to drive improvement in personal understanding as recited in claim 9, and further including a step of providing a cognitive self category tool of self-knowledge, wherein the software application generates questions to the user for answering thereof for determination of cognitive ability of the user.

11. The self-development method facilitated by the software application operable to drive improvement in personal understanding as recited in claim 10, and further including a step of providing an individual need category tool of self-knowledge, wherein questions are provided to the user on a need category, wherein responses thereto are recorded in the personal database and the software application determines a development value of the individual need category tool of self-knowledge.

12. The self-development method facilitated by the software application operable to drive improvement in personal understanding as recited in claim 11, and further including a step of providing an individual analysis category tool of self-knowledge, wherein questions are provided to the user on the individual analysis to determine past happenings to the user, wherein responses thereto are recorded in the personal database and the software application determines a development value of the individual analysis category tool of self-knowledge.

13. The self-development method facilitated by the software application operable to drive improvement in personal understanding as recited in claim 12, and further including a step of providing a fashion identity category tool of self-knowledge, wherein the user employs the software application to dress the personal three-dimensional avatar a plurality of times, wherein the user can select from a multitude of clothing items to dress the personal three-dimensional avatar.
